(19) United States
(12) Patent Application Publication
(10) Pub. No.: US 2023/0182122 A1
(43) Pub. Date: Jun. 15, 2023

(54) USE OF MAGNETIC MESOPOROUS POLY(IONIC LIQUID) INTERFACIAL CATALYST IN HYDROGENATION REACTION AND PREPARATION OF BIODIESEL

(71) Applicant: Qufu Normal University, Qufu City (CN)

(72) Inventors: Anguo Ying, Qufu City (CN); Shengnan Li, Qufu City (CN); Xiaotong Lu, Qufu City (CN); Qi Liu, Qufu City (CN); Yujing Liu, Qufu City (CN); Zhongqiu Liu, Qufu City (CN)

(21) Appl. No.: 17/872,385

(22) Filed: Jul. 25, 2022

(30) Foreign Application Priority Data
Dec. 14, 2021 (CN) 202111521332.0

(51) Int. Cl. B01J 31/28; C07C 5/03; B01J 23/745; B01J 31/22; B01J 31/40; C11C 3/04

(52) U.S. Cl. CPC B01J 31/28; B01J 23/745; B01J 31/2295; B01J 31/4069; C07C 5/03; C11C 3/04; C07C 2523/745; C07C 2531/22; C07C 2531/28

(57) ABSTRACT

The disclosure provides use of an efficient, recyclable, green and friendly catalyst to realize a method of hydrogenation of an unsaturated alkene, and a method for preparing biodiesel through the transesterification of soybean oil with ethanol. The method of hydrogenation of the unsaturated alkene comprises performing a hydrogenation reaction of an unsaturated alkene at ambient temperature and atmospheric pressure by using a CO₂ and magnetic dual-responsive mesoporous poly(ionic liquid) as a catalyst I, and using n-hexane and water as a solvent, to obtain a corresponding saturated alkane. The method for preparing biodiesel through transesterification of soybean oil with ethanol comprises performing a transesterification reaction of soybean oil with ethanol at a temperature of 25-90° C. and atmospheric pressure by using a CO₂ and magnetic dual-responsive mesoporous poly(ionic liquid) as a catalyst II, to obtain the biodiesel.

### CROSS REFERENCE TO RELATED APPLICATION

This patent application claims the benefit and priority of Chinese Patent Application No. 202111521332.0, entitled “Use of magnetic mesoporous poly(ionic liquid) interfacial catalyst in hydrogenation reaction and preparation of biodiesel,” filed on Dec. 14, 2021, the disclosure of which is incorporated by reference herein in its entirety as part of the present application.

### TECHNICAL FIELD

The present disclosure relates to an environmentally-friendly, efficient, and recyclable mesoporous poly(ionic liquid) catalyst with a CO₂ and magnetic double response, which is prepared by a template-free process, a method for preparing a corresponding saturated alkane through hydrogenation of an unsaturated alkene at ambient temperature and atmospheric pressure by using n-hexane and water as a solvent in the presence of the above catalyst, and a method for preparing biodiesel through a transesterification reaction between soybean oil and ethanol at a temperature of 25-90° C. and atmospheric pressure in the presence of the above catalyst.

### BACKGROUND ART

Pickering emulsions, being stable emulsions formed by adsorbing innumerable solid particles in the micro- to nanometer range as stabilizers onto the liquid-liquid interface, are well known for their excellent stability and have favorable environmental friendliness. Pickering emulsions have potential applications in different fields, such as catalysis, food, cosmetics, medicine, materials, and other fields. Especially in catalysis, Pickering emulsion interfacial catalyst has significantly improved the catalytic reaction efficiency and shortened the reaction time. This Pickering emulsion system makes the biphasic reaction greener and more environmentally friendly, so it is commonly used in various reactions such as hydrogenation, epoxidation, and hydroformylation.

In addition, mesoporous materials, with a pore size in the range of 2-50 nm and a well-developed pore-structure and functionalized moiety, have properties such as adsorption and storage. The mesoporous structure can provide unique nanoscale effects and adjustable pore size distribution and increase the number of accessible active sites, which is advantageous for improving the catalysis effect in the reactions. Consequently, mesoporous materials are often used as high-capacity electrodes, catalytic supports, energy storage devices, and so on. Additionally, biodiesel, usually referring to fatty acid methyl ester or fatty acid ethyl ester formed by esterification of vegetable oil, animal oil, waste oil, etc. with methanol or ethanol, is a typical green energy with perfect environmental protection performance, a wide range of raw material sources, and renewable and other characteristics. The development of biodiesel is of great strategic significance to the sustainable development of the economy and conservation and reuse of energy. Carbon dioxide, as an abundant, nonhazardous, and low-cost gas, can interact with certain functional groups for modification. Therefore, the inventors introduce 2,2,6,6-tetramethyl-4-piperidyl methacrylate (TEMPA) as a CO₂-responsive functional group to accelerate the demulsification process, and fabricate environmentally-friendly, efficient and recyclable mesoporous poly(ionic liquid) catalyst with a CO₂ and magnetic double response by a template-free process. After the reactions by using the mesoporous poly(ionic liquid) catalyst, the demulsification process is accelerated by an external magnetic force and blowing CO₂, which is conducive to quickly separating the catalyst from the product solution, and thereby further conducive to the recycling of the catalyst.

### SUMMARY

An object of the present disclosure is to replace the method for preparing saturated alkane compounds through catalytic hydrogenation reaction, and the method for preparing biodiesel through catalytic transesterification reaction by using traditional catalysts, and to provide an environmentally-friendly, efficient and recyclable mesoporous poly(ionic liquid) catalyst with a CO₂ and magnetic double response, which makes it possible to catalyze hydrogenation reactions at ambient temperature and atmospheric pressure by using n-hexane and water as a solvent, and to catalyze a reaction between soybean oil and ethanol at atmospheric pressure to realize the synthesis of biodiesel.

According to the present disclosure, a method for preparing a saturated alkane through hydrogenation reaction of an unsaturated alkene with hydrogen comprises performing a hydrogenation reaction of an unsaturated alkene with hydrogen at ambient temperature and atmospheric pressure for 10-20 min by using a CO₂ and magnetic dual-responsive mesoporous poly(ionic liquid) as a catalyst I and using n-hexane and water as a solvent, to obtain a corresponding saturated alkane. In some embodiments, the catalyst I has a schematic structure as shown in FIG. 3. A method for preparing biodiesel from soybean oil and ethanol comprises performing a transesterification reaction of soybean oil with ethanol at a temperature of 25-90° C. and atmospheric pressure by using a CO₂ and magnetic dual-responsive mesoporous poly(ionic liquid) as a catalyst II, to obtain the biodiesel. In some embodiments, the catalyst II has a schematic structure as shown in FIG. 4.

In some embodiments, a volume ratio of n-hexane to water is 1:1, 1:2, or 2:1.

In some embodiments, a molar ratio of ethanol to soybean oil is in a range of 5:1 to 19:1.

In some embodiments, a molar ratio of the catalyst I to the unsaturated alkene is in a range of 0.007:1 to 0.012:1.

In some embodiments, a molar ratio of the catalyst II to soybean oil is in a range of 0.007:1 to 0.035:1.

In some embodiments, the hydrogenation reaction by using the catalyst I is performed at ambient temperature and atmospheric pressure.

In some embodiments, the transesterification reaction by using the catalyst II is performed at a temperature of 25-90° C.

In some embodiments, the unsaturated alkene is selected from the group consisting of styrene, phenylacetylene, allylbenzene, cyclohexene, n-butyl acrylate, butyl methacrylate, 1-octene, and 1-dodecene.

In some embodiments, the method for preparing a saturated alkane further comprises, after the hydrogenation reaction, separating the catalyst I from a first product solution by an external magnetic force and blowing CO₂, and pouring out a first clear liquid from the first product solution to obtain a first product; and washing the catalyst I with methanol to obtain a washed catalyst I, and vacuum-drying the washed catalyst I at a temperature of 60° C. for 5 hours, such that the catalyst I is recyclable for more than one time.

In some embodiments, the method for preparing biodiesel further comprises, after the transesterification reaction, separating the catalyst II from a second product solution by an external magnetic force and blowing CO₂, and pouring out a second clear liquid from the second product solution to obtain a second product; and washing the catalyst II with methanol to obtain a washed catalyst II, and vacuum-drying the washed catalyst II at a temperature of 60° C. for 5 hours, such that the catalyst II is recyclable for more than one time.

In some embodiments, the method of hydrogenation of an unsaturated alkene and the method for preparing biodiesel through transesterification of soybean oil with ethanol by using a CO₂ and magnetic dual-responsive mesoporous poly(ionic liquid) as a catalyst according to the present disclosure could be implemented by the following procedures:

Preparation of the CO₂ and magnetic dual-responsive mesoporous poly(ionic liquid) catalyst:

Equimolar of 2-bromoethyl acrylate and triethylenediamine are dissolved in methanol to obtain a first mixed solution, and under the protection of nitrogen, the first mixed solution is heated to 55° C. and kept at 55° C. for 24 hours to obtain a product mixture. The product mixture is then concentrated at a reduced pressure, and vacuum-dried, to obtain a pale yellow viscous liquid, i.e., an ionic liquid. The structure of the prepared ionic liquid is confirmed by ¹H NMR (nuclear magnetic resonance). The ionic liquid monomer has a molecular formula as shown in FIG. 2.

The obtained ionic liquid, terminal alkene-modified Fe₃O₄@SiO₂, divinylbenzene, azobisisobutyronitrile, and 2,2,6,6-tetramethyl-4-piperidyl methacrylate (TEMPA) are dissolved in methanol to obtain a second mixed solution, and the second mixed solution is mechanically stirred at 70° C. for 6 hours (i.e., subjected to a reaction). After the reaction, the resulting reaction product is dried at 70° C. for 5 hours, to obtain a precursor with a mesoporous structure and good specific surface area, i.e., p(xTEMPA-yFDABCO-zDVB)@Fe₃O₄, wherein x, y, z represent the mole amount, respectively, which is confirmed by infrared spectrum and transmission electron microscopy (TEM).

For the catalyst I:

The precursor (0.15 g), palladium acetate (9 mg) and methanol (10 mL) are added into a reaction tube and intensely stirred at ambient temperature for 4 hours. NaBH₄ (8 mg) is then added thereto, and further stirred for another 2 hours. Finally, the resulting product mixture is centrifuged to obtain a solid. The solid is washed with methanol, and then dried at 80° C. for 4 hours, to obtain a catalyst I, i.e., Pd-p(xTEMPA-yFDABCO-zDVB)@Fe₃O₄, which is confirmed by XRD (X-ray diffraction), XPS (X-ray photoelectron spectroscopy) and TEM. The schematic structure thereof is shown in FIG. 3.

For the catalyst II:

The precursor (0.25 g), NaOH (0.1 g), methanol (10 mL) and water (2 mL) are added into a reaction tube, and intensely stirred at ambient temperature for 16 hours. The treated precursor is finally washed with methanol and water, and dried at 80° C. for 6 hours, to obtain a catalyst II, i.e. p(xTEMPA-y[FDABCO][OH]-zDVB)@Fe₃O₄, which is confirmed by XRD, infrared spectrum, and TEM. The schematic structure thereof is shown in FIG. 4.

Preparation of a saturated alkane product:

Pd-p(3TEMPA-FDABCO-2DVB)@Fe₃O₄ (20 mg), styrene (2 mmol), n-hexane (1 mL), and water (2 mL) are added into a reaction flask to obtain a first mixture, wherein the amount of styrene is 2 mmol, a molar ratio of the catalyst I to styrene is in a range of 0.007:1 to 0.012:1, and n-hexane and water are used as a solvent. The first mixture is subjected to a reaction at ambient temperature and atmospheric pressure for 10 min, and the reaction progress is tracked by gas chromatography. After the reaction, the catalyst I is separated from a first product solution by an external magnetic force and blowing CO₂, and a first clear liquid from the first product solution is poured out as a first product. The catalyst I is washed with methanol, and then vacuum-dried at 60° C. for 5 hours, which is used for the next batch of reaction. The catalyst I is recycled for 5 times, without significant decrease in yield.

Preparation of biodiesel:

p(xTEMPA-y[FDABCO][OH]-zDVB)@Fe₃O₄ (20 mg), soybean oil (1.145 mmol), and ethanol (8 mmol) are added into a reaction flask to obtain a second mixture, wherein a molar ratio of the catalyst II to soybean oil is in a range of 0.007:1 to 0.035:1. The second mixture is subjected to a transesterification reaction at a temperature of 25-90° C. and atmospheric pressure for 4 h, and the reaction progress is tracked by gas chromatography. After the reaction, the catalyst II is separated from a second product solution by an external magnetic force and CO₂, and a second clear liquid from the second product solution is poured out as a second product. The catalyst II is washed with methanol, and then vacuum-dried at 60° C. for 5 hours for the next batch of reaction. The catalyst II is recycled for 5 times, without significant decrease in yield.

### BRIEF DESCRIPTION OF THE DRAWINGS

The accompanying drawings, which are incorporated in and constitute a part of this description, illustrate embodiments consistent with the present disclosure, and together with the description serve to explain the principles of the present disclosure.

FIG. 1 shows a flow chart of the preparation of the magnetic mesoporous poly(ionic liquid) interfacial catalyst in one embodiment of the present disclosure;

FIG. 2 shows the molecular formula of the ionic liquid monomer in one embodiment of the present disclosure;

FIG. 3 shows a schematic structure of the catalyst Pd-p(xTEMPA-yFDABCO-zDVB)@Fe₃O₄ in one embodiment of the present disclosure; and

FIG. 4 shows a schematic structure of the catalyst p(xTEMPA-y[FDABCO][OH]-zDVB)@Fe₃O₄ in one embodiment of the present disclosure.

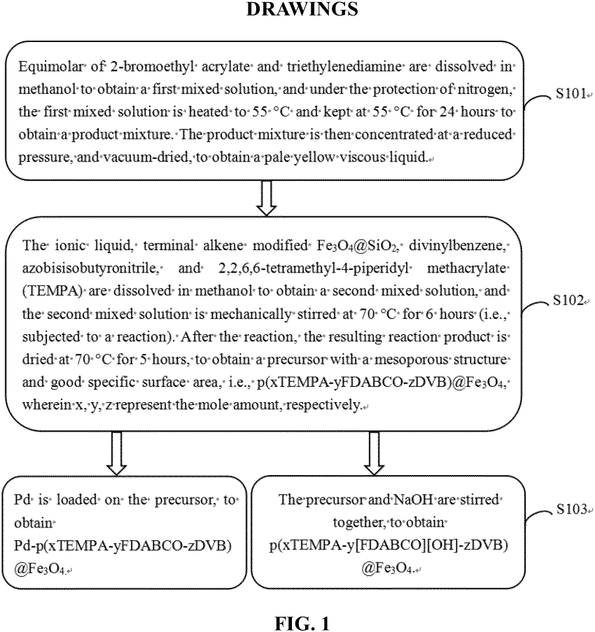

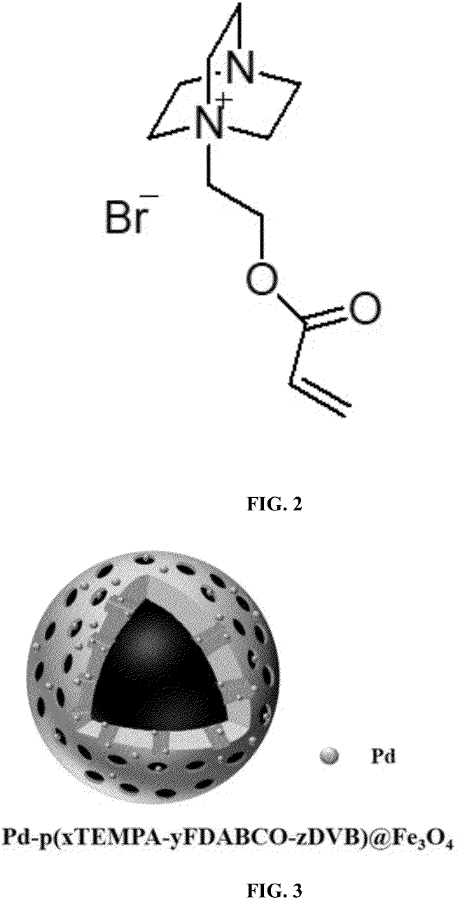

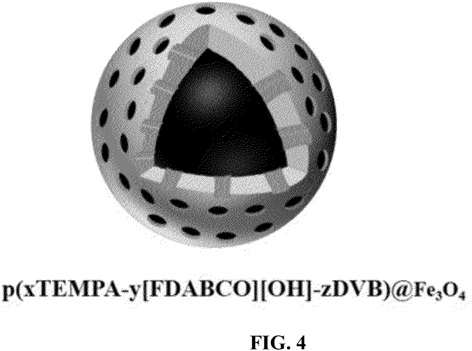

### DETAILED DESCRIPTION OF THE EMBODIMENTS

The present disclosure will be further described below in conjunction with examples. The examples of the present disclosure are only used to illustrate the technical solutions of the present disclosure and are not intended to limit the present disclosure.

### Example 1

Pd-p(3TEMPA-FDABCO-2DVB)@Fe₃O₄ (20 mg), styrene (2 mmol), n-hexane (1 mL), and water (2 mL) were added into a reaction tube, and intensely stirred to form a stable Pickering emulsion. H₂ was then circulated into the Pickering emulsion and reacted for 10 min. The reaction was monitored by gas chromatography. After the reaction, the catalyst was separated from a product solution by an external magnetic force and CO₂, and a clear liquid from the product solution was collected as the final product, with a conversion of 99%.

### Example 2

Pd-p(TEMPA-FDABCO-DVB)@Fe₃O₄ (20 mg), styrene (2 mmol), n-hexane (1 mL) and water (2 mL) were added into a reaction tube, and intensely stirred to form a stable Pickering emulsion. H₂ was then circulated into the Pickering emulsion and reacted for 10 min. The reaction was monitored by gas chromatography. After the reaction, the catalyst was separated from a product solution by an external magnetic force and CO₂, and a clear liquid from the product solution was collected as the final product, with a conversion of 60.59%.

### Example 3

Pd-p(2.5TEMPA-2.5FDABCO-DVB)@Fe₃O₄ (20 mg), styrene (2 mmol), n-hexane (1 mL) and water (2 mL) were added into a reaction tube, and intensely stirred to form a stable Pickering emulsion. H₂ was then circulated into the Pickering emulsion and reacted for 10 min. The reaction was monitored by gas chromatography. After the reaction, the catalyst was separated from a product solution by an external magnetic force and CO₂, and a clear liquid from the product solution was collected as the final product, with a conversion of 54.72%.

### Example 4

Pd-p(TEMPA-3FDABCO-2DVB)@Fe₃O₄ (20 mg), styrene (2 mmol), n-hexane (1 mL) and water (2 mL) were added into a reaction tube, and intensely stirred to form a stable Pickering emulsion. H₂ was then circulated into the Pickering emulsion and reacted for 10 min. The reaction was monitored by gas chromatography. After the reaction, the catalyst was separated from a product solution by an external magnetic force and CO₂, and a clear liquid from the product solution was collected as the final product, with a conversion of 80.77%.

### Example 5

Pd-p(3TEMPA-FDABCO-2DVB)@Fe₃O₄ (15 mg), styrene (2 mmol), n-hexane (1 mL) and water (2 mL) were added into a reaction tube, and intensely stirred to form a stable Pickering emulsion. H₂ was then circulated into the Pickering emulsion and reacted for 10 min. The reaction was monitored by gas chromatography. After the reaction, the catalyst was separated from a product solution by an external magnetic force and CO₂, and a clear liquid from the product solution was collected as the final product, with a conversion of 71%.

### Example 6

Pd-p(3TEMPA-FDABCO-2DVB)@Fe₃O₄ (20 mg), phenylacetylene (2 mmol), n-hexane (1 mL) and water (2 mL) were added into a reaction tube, and intensely stirred to form a stable Pickering emulsion. H₂ was then circulated into the Pickering emulsion and reacted for 15 min. The reaction was monitored by gas chromatography. After the reaction, the catalyst was separated from a product solution by an external magnetic force and CO₂, and a clear liquid from the product solution was collected as the final product, with a conversion of 91%.

### Example 7

Pd-p(3TEMPA-FDABCO-2DVB)@Fe₃O₄ (20 mg), allylbenzene (2 mmol), n-hexane (1 mL) and water (2 mL) were added into a reaction tube, and intensely stirred to form a stable Pickering emulsion. H₂ was then circulated into the Pickering emulsion and reacted for 10 min. The reaction was monitored by gas chromatography. After the reaction, the catalyst was separated from a product solution by an external magnetic force and CO₂, and a clear liquid from the product solution was collected as the final product, with a conversion of 99%.

### Example 8

Pd-p(3TEMPA-FDABCO-2DVB)@Fe₃O₄ (20 mg), cyclohexene (2 mmol), n-hexane (1 mL) and water (2 mL) were added into a reaction tube, and intensely stirred to form a stable Pickering emulsion. H₂ was then circulated into the Pickering emulsion and reacted for 20 min. The reaction was monitored by gas chromatography. After the reaction, the catalyst was separated from a product solution by an external magnetic force and CO₂, and a clear liquid from the product solution was collected as the final product, with a conversion of 88%.

### Example 9

Pd-p(3TEMPA-FDABCO-2DVB)@Fe₃O₄ (20 mg), n-butyl acrylate (2 mmol), n-hexane (1 mL) and water (2 mL) were added into a reaction tube, and intensely stirred to form a stable Pickering emulsion. H₂ was then circulated into the Pickering emulsion and reacted for 10 min. The reaction was monitored by gas chromatography. After the reaction, the catalyst was separated from a product solution by an external magnetic force and CO₂, and a clear liquid from the product solution was collected as the final product, with a conversion of 99%.

### Example 10

Pd-p(3TEMPA-FDABCO-2DVB)@Fe₃O₄ (20 mg), butyl methacrylate (2 mmol), n-hexane (1 mL) and water (2 mL) were added into a reaction tube, and intensely stirred to form a stable Pickering emulsion. H₂ was then circulated into the Pickering emulsion and reacted for 10 min. The reaction was monitored by gas chromatography. After the reaction, the catalyst was separated from a product solution by an external magnetic force and CO₂, and a clear liquid from the product solution was collected as a final product, with a conversion of 99%.

### Example 11

Pd-p(3TEMPA-FDABCO-2DVB)@Fe₃O₄ (20 mg), 1-octene (2 mmol), n-hexane (1 mL) and water (2 mL) were added into a reaction tube, and intensely stirred to form a stable Pickering emulsion. H₂ was then circulated into the Pickering emulsion and reacted for 15 min. The reaction was monitored by gas chromatography. After the reaction, the catalyst was separated from a product solution by an external magnetic force and CO₂, and a clear liquid from the product solution was collected as a final product, with a conversion of 99%.

### Example 12

Pd-p(3TEMPA-FDABCO-2DVB)@Fe₃O₄ (20 mg), 1-dodecene (2 mmol), n-hexane (1 mL) and water (2 mL) were added into a reaction tube, and intensely stirred to form a stable Pickering emulsion. H₂ was then circulated into the Pickering emulsion and reacted for 20 min. The reaction was monitored by gas chromatography. After the reaction, the catalyst was separated from a product solution by an external magnetic force and CO₂, and a clear liquid from the product solution was collected as a final product, with a conversion of 99%.

### Example 13

Pd-p(3TEMPA-FDABCO-2DVB)@Fe₃O₄ (20 mg), styrene (2 mmol), n-hexane (1 mL) and water (2 mL) were added into a reaction tube, and intensely stirred to form a stable Pickering emulsion. H₂ was then circulated into the Pickering emulsion and reacted for 10 min. The reaction was monitored by gas chromatography. After the reaction, the catalyst was separated from a product solution by an external magnetic force and CO₂, and a clear liquid from the product solution was collected as a final product, with a conversion of 99%. The catalyst was recycled for 5 times, without significant decrease in yield, as shown in Table 1.

### Example 14

p(3TEMPA-[FDABCO][OH]-2DVB)@Fe₃O₄ (20 mg), soybean oil (1.145 mmol), and ethanol (10 mmol) were added into a reaction tube, and intensely stirred to form a stable Pickering emulsion. The Pickering emulsion was subjected to a reaction at 65° C. and atmospheric pressure for 4 h. The reaction was monitored by gas chromatography. After the reaction, the catalyst was separated from a product solution by an external magnetic force and CO₂, and a clear liquid of the product solution is subjected to an extraction with n-hexane, obtaining biodiesel, with a yield of 80.84%.

### Example 15

p(3TEMPA-[FDABCO][OH]-2DVB)@Fe₃O₄ (20 mg), soybean oil (1.145 mmol), and ethanol (10 mmol) were added into a reaction tube, and intensely stirred to form a stable Pickering emulsion. The Pickering emulsion was subjected to a reaction at 25° C. and atmospheric pressure for 4 h. The reaction was monitored by gas chromatography. After the reaction, the catalyst was separated from a product solution by an external magnetic force and CO₂, and a clear liquid of the product solution is subjected to an extraction with n-hexane, obtaining biodiesel, with a yield of 75.87%.

### Example 16

p(3TEMPA-[FDABCO][OH]-2DVB)@Fe₃O₄ (20 mg), soybean oil (1.145 mmol), and ethanol (10 mmol) were added into a reaction tube, and intensely stirred to form a stable Pickering emulsion. The Pickering emulsion is subjected to a reaction at 90° C. and atmospheric pressure for 4 h. The reaction was monitored by gas chromatography. After the reaction, the catalyst was separated from a product solution by an external magnetic force and CO₂, and a clear liquid of the product solution was subjected to an extraction with n-hexane, obtaining biodiesel, with a yield of 91.55%.

### Example 17

p(TEMPA-[FDABCO][OH]-4DVB)@Fe₃O₄ (20 mg), soybean oil (1.145 mmol), and ethanol (10 mmol) were added into a reaction tube, and intensely stirred to form a stable Pickering emulsion. The Pickering emulsion was subjected to a reaction at 65° C. and atmospheric pressure for 4 h. The reaction was monitored by gas chromatography. After the reaction, the catalyst was separated from a product solution by an external magnetic force and CO₂, and a clear liquid of the product solution was subjected to an extraction with n-hexane, obtaining biodiesel, with a yield of 41.45%.

### Example 18

p(3TEMPA-[FDABCO][OH]-2DVB)@Fe₃O₄ (20 mg), soybean oil (1.145 mmol), and ethanol (5 mmol) were added into a reaction tube, and intensely stirred to form a stable Pickering emulsion. The Pickering emulsion was subjected to a reaction at 65° C. and atmospheric pressure for 4 h. The reaction was monitored by gas chromatography. After the reaction, the catalyst was separated from a product solution by an external magnetic force and CO₂, and a clear liquid of the product solution was subjected to an extraction with n-hexane, obtaining biodiesel, with a yield of 47.91%.

### Example 19

p(3TEMPA-[FDABCO][OH]-2DVB)@Fe₃O₄ (10 mg), soybean oil (1.145 mmol), and ethanol (10 mmol) were added into a reaction tube, and intensely stirred to form a stable Pickering emulsion. The Pickering emulsion was subjected to a reaction at 65° C. and atmospheric pressure for 4 h. The reaction was monitored by gas chromatography. After the reaction, the catalyst was separated from a product solution by an external magnetic force and CO₂, and a clear liquid of the product solution was subjected to an extraction with n-hexane, obtaining biodiesel, with a yield of 79.42%.

### Example 20

p(3TEMPA-[FDABCO][OH]-2DVB)@Fe₃O₄ (20 mg), soybean oil (1.145 mmol), and ethanol (19 mmol) were added into a reaction tube, and intensely stirred to form a stable Pickering emulsion. The Pickering emulsion was subjected to a reaction at 65° C. and atmospheric pressure for 4 h. The reaction was monitored by gas chromatography. After the reaction, the catalyst was separated from a product solution by an external magnetic force and CO₂, and a clear liquid of the product solution was subjected to an extraction with n-hexane, obtaining biodiesel, with a yield of 65.43%.

### Example 21

p(3TEMPA-[FDABCO][OH]-2DVB)@Fe₃O₄ (20 mg), soybean oil (1.145 mmol), and ethanol (10 mmol) were added into a reaction tube, and intensely stirred to form a stable Pickering emulsion. The Pickering emulsion was subjected to a reaction at 65° C. and atmospheric pressure for 4 h. The reaction was monitored by gas chromatography. After the reaction, the catalyst was separated from a product solution by an external magnetic force and CO₂, and a clear liquid of the product solution was subjected to an extraction with n-hexane, obtaining biodiesel, with a yield of 80.84%. The catalyst was recycled for 5 times, without significant decrease in yield, as shown in Table 2.

TABLE 1

| Times | Temperature (°C) | Time for reaction (min) | Conversions(%) |
|---|---|---|---|
| 1 | 25 | 10 | 99 |
| 2 | 25 | 10 | 97 |
| 3 | 25 | 10 | 93 |
| 4 | 25 | 10 | 94 |
| 5 | 25 | 10 | 90 |

TABLE 2

| Times | Temperature (°C) | Time for reaction (h) | Yield(%) |
|---|---|---|---|
| 1 | 65 | 4 | 80.84 |
| 2 | 65 | 4 | 80.34 |
| 3 | 65 | 4 | 78.67 |
| 4 | 65 | 4 | 76.37 |
| 5 | 65 | 4 | 50.23 |

It should be noted that the above summary and the specific embodiments of the present disclosure are intended to prove the practical application of the technical solutions according to the present disclosure and should not be construed as limiting the scope of the present disclosure. Those skilled in the art could make various modifications, equivalent substitutions, or improvements within the spirit and principles of the present disclosure. The scope of the present disclosure is accorded with the appended claims.

What is claimed is:

1. A method of hydrogenation of an unsaturated alkene, comprising: performing a hydrogenation reaction of an unsaturated alkene at ambient temperature and atmospheric pressure by using a CO₂ and magnetic dual-responsive mesoporous poly(ionic liquid) as a catalyst I, and using n-hexane and water as a solvent, to obtain a corresponding saturated alkane.

2. The method of claim 1, wherein the catalyst I has a large specific surface area of 51.22-272.49 m²·g⁻¹ and a good pore size distribution, and is prepared by a template-free process comprising introducing 2,2,6,6-tetramethyl-4-piperidyl methacrylate (TEMPA) monomer and a terminal alkene-modified Fe₃O₄@SiO₂.

3. The method of claim 1, wherein a volume ratio of n-hexane to water is 1:1, 1:2, or 2:1.

4. The method of claim 1, wherein a molar ratio of the catalyst I to the unsaturated alkene is in a range of 0.007: 1 to 0.012: 1.

5. The method of claim 1, wherein the hydrogenation reaction by using the catalyst I is performed at ambient temperature and atmospheric pressure.

6. The method of claim 1, wherein the unsaturated alkene is selected from a group consisting of styrene, phenylacetylene, allylbenzene, cyclohexene, n-butyl acrylate, butyl methacrylate, 1-octene, and 1-dodecene.

7. The method of claim 1, further comprising:
after the hydrogenation reaction, separating the catalyst I from a first product solution by an external magnetic force and blowing CO₂, and pouring out a first clear liquid from the first product solution to obtain a first product; and
washing the catalyst I with methanol to obtain a washed catalyst I, and vacuumdrying the washed catalyst I at 60° C. for 5 hours, such that the catalyst I is recyclable for more than one time.

8. The method of claim 2, wherein the unsaturated alkene is selected from a group consisting of styrene, phenylacetylene, allylbenzene, cyclohexene, n-butyl acrylate, butyl methacrylate, 1-octene, and 1-dodecene.

9. The method of claim 3, wherein the unsaturated alkene is selected from a group consisting of styrene, phenylacetylene, allylbenzene, cyclohexene, n-butyl acrylate, butyl methacrylate, 1-octene, and 1-dodecene.

10. The method of claim 4, wherein the unsaturated alkene is selected from a group consisting of styrene, phenylacetylene, allylbenzene, cyclohexene, n-butyl acrylate, butyl methacrylate, 1-octene, and 1-dodecene.

11. A method for preparing biodiesel through transesterification of soybean oil with ethanol, comprising: performing a transesterification reaction of soybean oil with ethanol at a temperature of 25-90° C. and atmospheric pressure by using a CO₂ and magnetic dual-responsive mesoporous poly(ionic liquid) as a catalyst II, to obtain the biodiesel.

12. The method of claim 11, wherein the catalyst II has a large specific surface area of 51.22-272.49 m²·g⁻¹ and a good pore size distribution, and is prepared by a template-free process comprising introducing 2,2,6,6-tetramethyl-4-piperidyl methacrylate (TEMPA) monomer and a terminal alkene-modified Fe₃O₄@SiO₂.

13. The method of claim 11, wherein a molar ratio of ethanol to soybean oil is in a range of 5:1 to 19:1.

14. The method of claim 11, wherein the transesterification reaction by using the catalyst II is performed at a temperature of 25-90° C.

15. The method of claim 11, wherein a molar ratio of the catalyst II to soybean oil is in a range of 0.007: 1 to 0.035:1.

16. The method of claim 11, further comprising:
after the transesterification reaction, separating the catalyst II from a second product solution by an external magnetic force and blowing CO₂, and pouring out a second clear liquid from the second product solution to obtain a second product; and
washing the catalyst II with methanol to obtain a washed catalyst II, and vacuumdrying the washed catalyst II at 60° C. for 5 hours, such that the catalyst II is recyclable for more than one time.
